(19) United States
(12) Patent Application Publication
(10) Pub. No.: US 2005/0256314 A1
(43) Pub. Date: Nov. 17, 2005

(54) Process employing controlled crystallization in forming crystals of a pharmaceutical

(76) Inventors: Soojin Kim, West Orange, NJ (US); Chenkou Wei, Princeton Junction, NJ (US); Mark D. Lindrud, Basking Ridge, NJ (US); Hyei-Jha Chung, Plainsboro, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/119,551

(22) Filed: May 2, 2005

Related U.S. Application Data
(60) Provisional application No. 60/568,043, filed on May 4, 2004.
(60) Provisional application No. 60/607,533, filed on Sep. 7, 2004.

(51) Int. Cl. C07D 263/34

(52) U.S. Cl. 548/236; 23/295 R

(57) ABSTRACT

A process is provided which employs reactive controlled crystallization to produce drug substance having desirable crystal properties which process involves providing reactants A and B in liquid or solution form and adding reactant B to reactant A using a cubic or incremental addition technique to control extent of reaction and thus crystallization kinetics, including supersaturation and nucleation, to produce crystals of drug substance which are generally larger, better quality and with few fines and narrow particle size distribution than normally obtainable employing prior art crystallization techniques. In addition, crystals of drug substance produced by the above process is also provided.

### REFERENCE TO OTHER APPLICATION

The present application takes priority from U.S. provisional application Nos. 60/568,043 filed May 4, 2004, and 60/607,533 filed Sep. 7, 2004, the disclosures of which are incorporated herein by reference.

### FIELD OF THE INVENTION

The present invention relates to a process for forming crystals of a salt of a pharmaceutical by reactive controlled crystallization employing a cubic or incremental reactant addition technique to control extent of reaction and thus crystallization kinetics and to crystals of a pharmaceutical produced by such process.

### BACKGROUND OF THE INVENTION

Crystallization is a critical operation in the manufacture of pharmaceutical compounds. The crystallization process as part of the synthesis of an active pharmaceutical ingredient (API) affects the API crystal properties such as purity, polymorphic form, particle size and habit. Optimization of the crystallization process is important for API product quality as well as for process efficiency and high yield.

Crystal properties also significantly impact the downstream processing. For example, excess fines or wide particle size distribution may cause slow filtration and inefficient drying which may be a major bottleneck of the entire process, necessitating modification of the crystallization process to produce the type of particles that facilitate downstream processing.

Another important aspect of crystallization development involves particle engineering to obtain desired particle size or habit to meet the biopharmaceutical performance requirements. For insoluble or dissolution-limited drug substances, small particle size is necessary to maximize surface area to enhance bioavailability. Particle uniformity may be important to homogeneity of blend or granulation during formulation and consistent dosage of product. In addition, API crystal properties such as particle size distribution, habit and surface properties have large impact on the bulk powder properties which affect formulation operations such as blending, granulation, and compaction. Therefore, having consistent and optimal API physical properties is essential for the development of formulation processes to produce consistent and reliable product.

Design of crystallization processes is aimed at achieving drug substances with the desired characteristics in consistently high quality. On the other hand, preserving the crystals' quality and key physical properties throughout the downstream processing steps—such as filtration, drying, and delumping—may be a challenging task. For example, undesirable form change, particle size reduction or agglomeration may arise as a result of the downstream processing and cause poor product performance. For some cases, monitoring key particle properties during the processing steps following the crystallization may be necessary and would allow identification of the steps that cause adverse changes and help in implementing corrective measures.

The crystallization process employed could be especially important for a drug whose physical properties including crystal purity, polymorphic form, particle size and habit, have a strong effect on the formulation and drug product performance. Thus, a controlled crystallization process to produce optimal crystal properties that facilitate filtration, drying and powder handling that would preserve the quality of API crystals to achieve consistently excellent formulation characteristics and drug product performance would indeed be a welcomed addition to the pharmaceutical industry.

U.S. provisional application No. 60/568,043 filed May 4, 2004 from which the present application claims priority relates to a process for preparing the HIV protease inhibitor atazanavir bisulfate (also referred to as atazanavir sulfate) employing a reactive controlled crystallization technique, namely a modified cubic crystallization method based on volume of reactant added as opposed to uncontrolled crystallization process described in U.S. Pat. No. 6,087,383. The crystals of atazanavir bisulfate obtained by reactive controlled crystallization are generally larger and are of better quality than those obtained employing prior art procedures involving addition of sulfuric acid to a solution of atazanavir free base suspended in ethanol which causes the free base to dissolve and react to form the bisulfate salt. Crystallization of such bisulfate is initiated by seeding and subsequently adding heptanes as antisolvent, and the crystallization proceeds in an uncontrolled manner. The filtration process is slow with inefficient washing, and the resulting wet cake is highly compressible due to excess fines and wide particle size distribution caused by uncontrolled nucleation and crystallization. When dried, the wet cake compacts into hard lumps and requires extensive milling operation for further processing.

U.S. provisional application 60/572,397 filed May 19, 2004 discloses treating the Schiff's base 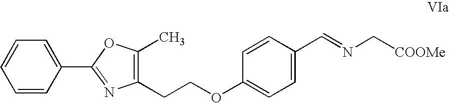
with an acid-salt forming reagent such as trimethylchlorosilane in the presence of an alcohol such as methanol, to form the hydrochloride acid salt IIa 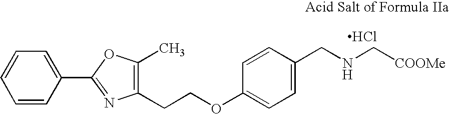
It is further disclosed that the optimal addition rate for the acid-salt forming reagent trimethylchlorosilane is a cubic addition profile for maximizing removal of organic contaminants.

### BRIEF DESCRIPTION OF THE INVENTION

In accordance with the present invention, a process is provided for forming crystals of a salt of a pharmaceutical by means of controlled reactive crystallization, which process includes the steps of
a) providing a first reactant in the form of a liquid;
b) providing a second reactant in the form of a liquid; and
c) adding the second reactant to the first reactant incrementally to form crystals.

In a preferred embodiment of the invention, the first reactant will be in the form of a free base or free acid of the pharmaceutical salt and the second reactant will be an acid or a base.

In carrying out the reactive controlled crystallization technique of the invention, the second reactant is added at a very slow rate initially and at an increasing rate according to the following equation

$$V = {V}_{total} \times {( \frac{t}{{t}_{total}} )}^{3}$$

where
V=Volume of second reactant added during the elapsed time period t
Vₜₒₜₐₗ=Total volume of second reactant for 100% reaction conversion
t=Elapsed time in crystallization
tₜₒₜₐₗ=Total crystallization time or total time for second reactant charging

Formation of crystals may be enhanced by adding seeds of crystals of the pharmaceutical salt to one of the reactants or to the reaction mixture of the first and second reactants after a portion of the second reactant (typically less than about 15% of total) is added.

Total crystallization time may be as short as 1 hour and as long as desired. Typically 2-8 hour of total addition time is effective. The longer the addition time, the slower the crystallization rate, and generally the larger the crystals obtained.

The process of the invention may be employed in preparing crystals of the HIV protease inhibitor atazanavir bisulfate as disclosed.

U.S. provisional application No. 60/568,043 filed May 4, 2004, the disclosure of which is incorporated herein by reference, discloses that crystals of the bisulfate salt of atazanavir bisulfate are formed by a process which employs the modified cubic crystallization technique (described herein) wherein sulfuric acid is added at an increasing rate according to a cubic equation (as described hereinafter), and includes the steps of reacting a solution of atazanavir free base in an organic solvent (in which the atazanavir bisulfate salt is substantially insoluble, such as acetone, a mixture of acetone and N-methylpyrrolidone, ethanol, or a mixture of ethanol and acetone) with a first portion of concentrated sulfuric acid in an amount to react with less than about 15%, preferably less than about 12%, by weight of the atazanavir free base, adding seeds of atazanavir bisulfate Form A crystals to the reaction mixture, and as crystals of atazanavir bisulfate form, adding additional concentrated sulfuric acid in multiple stages at increasing rates according to the cubic equation to effect formation of Form A crystals.

The process of the invention may also be employed for preparing crystals of the HCl salt (or other salt) of the structure 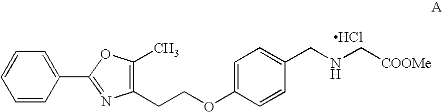
(hereinafter also referred to as the PPAR α/γ dual agonist intermediate)
by means of controlled reactive crystallization, which includes the steps of
a) preparing a solution of the free base of the structure 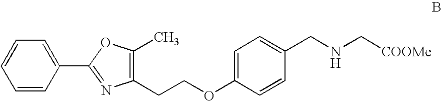
   (hereinafter also referred to as the PPAR free base)
   dissolved in a solvent in which the HCl salt (or other salt) of said free base is substantially insoluble such as ethyl acetate and premixing with methanol which serves as another reactant in the reaction;
b) adding chlorotrimethylenesilane incrementally to effect formation crystals of HCl salt (PPAR α/γ dual agonist intermediate); and
c) drying the crystals of HCl salt.

Crystal formation may be enhanced by adding seeds of the HCl salt of the free base to the solution of the free base.

The chlorotrimethylenesilane is added at an increasing rate according to the following cubic equation set out herein.

The above salt of the free acid (PPAR α/γ dual agonist intermediate) is employed as an intermediate in the preparation of compounds employed in treating Type II diabetes and dyslipidemia as disclosed in U.S. Pat. No. 6,414,002, the disclosure of which is incorporated herein by reference.

Crystallization of the free base B preferably involves an HCl salt crystallization by a reaction between the free base B and chlorotrimethylsilane in presence of methanol, employing a molar equivalent of chlorotrimethylsilane within the range from about 1 to about 1.2. The free base B is dissolved preferably in ethyl acetate/methanol (from 15:1 to 20:1 volume ratio). Preferably 1-1.2 or more molar equiv. of chlorotrimethylsilane is added to the free base solution incrementally. It is preferred to add chlorotrimethylsilane at a very slow rate initially and at increasing rate as crystallization proceeds. Seeding is preferred for better control of crystallization and can be done before chlorotrimethylsilane addition. Crystals are formed as a result of the HCl salt formation which crystallizes out in ethyl acetate.

Crystallization by this technique produces initially a thin slurry gradually increasing in solid mass as the addition progresses, whereas the crystallization by conventional methods (using uncontrolled addition) produces fast precipitation of large amount of solids that results in a thick and unstirrable slurry. The crystals from the cubic addition are well-defined and larger and produce less-compressible wet cake with good filtration and wash efficiency which also facilitate drying and powder handling.

In addition, in accordance with the present invention, crystals of a salt of a pharmaceutical prepared by the process as described above are also provided.

Finally, crystals of the HCl salt (or other salt) of the structure 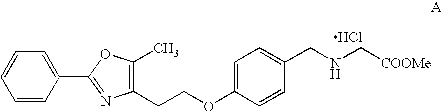
prepared by the process as defined above are provided.

The process of the invention employing controlled crystallization using cubic or incremental addition technique to control the extent of reaction and thus crystallization kinetics to produce optimal crystals of drug product is applicable to any reactive crystallization involving reactions such as
1) Acid+Base→salt crystals; or
2) A+B→crystal product; or
3) A+B+C→crystal product
   where A (including acids) and B (including bases) are liquids or are dissolved in separate solvents to form solutions, C (which may or may not be necessary) may be premixed with A or B, and crystals precipitate out as a result of reaction.

By controlling the extent of reaction of A, B, and C using incremental addition of one of the reactants into the solution of other reactant, supersaturation is controlled within low limits (such as 1-15%) and nucleation is minimized. The resulting crystals are generally larger, are of better quality, and with fewer fines and narrow size distribution than those produced employing uncontrolled crystallization techniques.

The process of the invention employing the above-described controlled crystallization technique produce crystals of drug product having desired and consistent physical properties. The crystals obtained are generally larger, more well-defined with tight particle size distribution and fewer fines than obtained employing uncontrolled addition or constant addition rate crystallization techniques. The controlled crystallization technique (especially of the cubic addition) of the invention provides less compressible filter cake, which aids in effective cake deliquoring and washing, as well as providing a more easily dried product with excellent powder properties than obtained employing uncontrolled or constant addition rate crystallization techniques. The active pharmaceutical ingredient prepared by the process of the invention also facilitates formulation by improved bulk flowability, bulk density, and powder properties and handling.

### DETAILED DESCRIPTION OF THE INVENTION

It is well known that a fast change of supersaturation particularly in the initial stage of the crystallization process results in the formation of a large number of crystal nuclei and generally yields a poor quality non-uniform product (Mullin and Nyvlt, 1971, Chem. Eng. Sci., 26, 369). Growth rates increase with higher operating level of supersaturation; however, the increase in nucleation rate is more sensitive to the higher supersaturation level, and plays the dominant role in the formation of particles, especially fines. Keeping the working level of supersaturation low to keep the nucleation rate low significantly improves the uniformity of product.

The elemental reactions for the reactive crystallization process for salts such as atazanavir bisulfate or the PPAR α/γ dual agonist intermediate may be written as:
Free Base+Acid→Salt (solution)
Salt (solution)→Salt (crystal)
The extent of the reaction and thus the crystallization can be limited by limiting the amount of the acid accessible for reaction. By controlling the addition rate of the acid, a measure of control over the rate of reaction is obtained and thus control over the rate of salt formation in solution

$$\frac{ⅆ {C}_{Salt ( solution )}}{ⅆ t}$$

which is equal to the rate of supersaturation change

$$\frac{ⅆ Δ {C}_{Salt ( solution )}}{ⅆ t} ,$$

as shown in the simple kinetic expression:

$$\begin{matrix}\frac{ⅆ Δ {C}_{Salt ( solution )}}{ⅆ t} = \frac{ⅆ {C}_{Salt ( solution )}}{ⅆ t} \\ = {k}_{r} {C}_{FB} {C}_{Acid} - {k}_{n} A ( t ) Δ {C}_{Salt ( solution )}^{n} - {k}_{g} Δ {C}_{Salt ( solution )}^{g}\end{matrix}$$

where kᵣ, kₙ, and k_{g} are the reaction, nucleation, and growth rate constants, A(t) is the surface area, C_{Salt(solution)} and ΔC_{Salt(solution)} are the concentration and supersaturation of the salt in solution, and C_{FB} and C_{Acid} are the free base and acid concentration in solution.

For controlled crystallization, the free base, such as atazanavir or the PPAR free base, is first dissolved in a suitable solvent, and the supersaturation is managed by controlled acid addition (with crystal seeds present) using an incremental addition of acid to control the rate of reaction/crystallization.

In accordance with the present invention, further refinement of the controlled crystallization uses “cubic addition” wherein the acid (or base depending upon the nature of the other reactant) is added at an incremental amount at a variable rate, slow at first and gradually faster towards the end as the number of crystals and surface area available for growth increase. This crystallization protocol is designed to minimize nucleation rate and encourage particle growth onto crystal seeds that serve as nuclei.

The cubic method in a temperature controlled crystallization has been derived (Mullin and Nyvlt, 1971, supra; Jones and Mullin, 1974, Chem. Eng. Sci., 29, 105) as the following simplified equation:

$$T = {T}_{max} - ( {T}_{max} - {T}_{min} ) \times {( \frac{t}{{t}_{total}} )}^{3}$$

where T is a temperature at time t, Tₘₐₓ and Tₘᵢₙ are starting and ending temperatures for crystallization and tₜₒₜₐₗ is total crystallization time. Since the crystallization of atazanavir or the PPAR α/γ dual agonist free base B is controlled by the addition rate of sulfuric acid or chlorotrimethylsilane, the following cubic equation with respect to volume, similar to the above equation, is used:

$$V = {V}_{total} \times {( \frac{t}{{t}_{total}} )}^{3}$$

where V is the volume of sulfuric acid or chlorotrimethylsilane added during the elapsed time period t and Vₜₒₜₐₗ is total volume of sulfuric acid or chlorotrimethylsilane charge.

By controlling the crystallization rate using the above expression, nucleation is controlled within acceptable limits as the system maintains a constant low level of supersaturation. The slow initial acid or chlorotrimethylsilane flow rate has been shown to favor crystal growth over nucleation. Thus, as the surface area increases with particle size, the seed bed is able to accept the increasing acid flow rate without inducing secondary nucleation. The slow initial addition rate allows time for the crystals to grow larger, increasing the mean size. This cubic protocol is also consistent with a well-known observation that smaller crystals in general grow at lower rates compared to larger crystals. As the crystals grow, faster surface integration kinetics allows larger crystals to grow at higher growth rates (Mullin, 1993, Crystallization, 3^{rd} Ed., Butterworth-Heineman, Oxford, pubis.).

The crystal particle size and morphology are dependent on the addition rate of the acid (or base). This cubic crystallization protocol carried out over 6-8 hours provides relatively larger, more well-defined crystals, along with a narrower particle size range and fewer fines, than a constant addition rate crystallization. The cubic crystallization provides less compressible filter cake, which aids in effective cake deliquoring and washing, as well as giving a more easily dried product with excellent bulk powder handling properties.

The crystallization process employed in the process of the invention resolve the issues of wide particle size distribution, wet cake compressibility and filtration rate, wash efficiency, powder properties and formulation problems. The crystals produced by the cubic controlled addition crystallization protocol of the invention are more consistent in quality and size distribution and facilitate filtration, drying, and formulation than those produced employing uncontrolled crystallization.

In carrying out the process of the invention for preparing Form A crystals of atazanavir bisulfate salt, a modified cubic crystallization technique is employed wherein atazanavir free base is dissolved in an organic solvent in which the atazanavir bisulfate salt is substantially insoluble and includes acetone, a mixture of acetone and N-methylpyrrolidone, ethanol, a mixture of ethanol and acetone and the like, to provide a solution having a concentration of atazanavir free base within the range from about 6.5 to about 9.7% by weight, preferably from about 6.9 to about 8.1% by weight atazanavir free base.

The solution of atazanavir free base is heated at a temperature within the range from about 35 to about 55° C., preferably from about 40 to about 50° C., and reacted with an amount of concentrated sulfuric acid (containing from about 95 to about 100% H₂SO₄) to react with less than about 15% (including 0 to about 15%), preferably from about 5 to less than about 12%, more preferably from about 8 to about 10% by weight of the total atazanavir free base. Thus, the starting solution of atazanavir free base will be initially reacted with less than about 15%, preferably from about 5 to about 12%, by weight of the total amount of sulfuric acid to be employed. During the reaction, the reaction mixture is maintained at a temperature within the range from about 35 to about 55° C., preferably from about 40 to about 50° C.

The reaction is allowed to continue for a period from about 12 to about 60 minutes, preferably from about 15 to about 30 minutes.

The reaction mixture is seeded with crystals of Form A atazanavir bisulfate employing an amount of seeds within the range from about 0.1 to about 80% by weight, preferably from about 3 to about 8% by weight, based on the weight of atazanavir free base remaining in the reaction mixture while maintaining the reaction mixture at a temperature within the range from about 35 to about 55° C., preferably from about 40 to about 50° C.

The reaction is allowed to continue until crystallization begins. Thereafter, sulfuric acid is added in multiple stages at an increasing rate according to the cubic equation as described below to form atazanavir bisulfate which upon drying produces Form A crystals.

In carrying out the process of the invention for preparing crystals of the PPAR α/γ dual agonist HCl salt (or other salt) intermediate A, a modified cubic crystallization technique is employed wherein PPAR α/γ dual agonist free base B is dissolved in an organic solvent in which the free base is substantially insoluble and includes ethyl acetate, butyl acetate, and the like, to provide a solution having a concentration of free base within the range from about 5 to about 20% by weight, preferably from about 6 to about 10% by weight free base.

The solution of free base B is heated at a temperature within the range from about 35 to about 55° C., preferably from about 40 to about 50° C. and mixed with methanol (third reactant), and reacted with an amount of chlorotrimethylsilane to react with less than about 10% (including 0 to 10%), preferably from less than about 5% by weight of the total free base B. Thus, the starting solution of free base B will be initially reacted with less than about 10% (including 0 to about 10%), preferably less than 5 by weight of the total amount of chlorotrimethylsilane to be employed.

The PPAR α/γ dual agonist free base solution may be seeded with crystals of PPAR α/γ dual agonist salt intermediate A (prior to adding chlorotrimethylsilane) employing an amount of seeds within the range from about 0.01 to about 20% by weight, preferably from about 0.1 to about 8% by weight, based on the weight of free base while maintaining a temperature within the range from about 35 to about 55° C., preferably from about 40 to about 50° C.

The free base B is reacted with incremental portions of chlorotrimethylsilane (preferably total 1-1.2 molar equivalent to the free base) to continuously form the HCl salt crystals. It is preferred to add chlorotrimethylsilane at a very slow rate initially and at increasing rate according to the cubic equation as described herein. The addition of chlorotrimethylsilane may be done at continuously increasing rate or alternatively in several addition stages each with fixed but successively higher addition rate. During the reaction, the reaction mixture is maintained at a temperature within the range from about 35 to about 55° C., preferably from about 40 to about 50° C.

The crystal particle size and morphology of the salts formed are dependent on the addition rate of the sulfuric acid or chlorotrimethylsilane or other acid or base or other salt forming reactant, which determines the crystallization rate. It has been found that a modified “cubic” crystallization technique (sulfuric acid or chlorotrimethylsilane or other reactant added at an increasing rate according to the cubic equation) provides relatively larger, more well defined bisulfate salt or HCl salt (or other salt) crystals, along with a narrower particle size range and fewer fines, than a constant addition rate crystallization. The slow initial sulfuric acid or chlorotrimethylsilane flow rate has been shown to favor crystal growth over secondary nucleation. Thus, as the surface area increases with particle size, the seed bed is able to accept the increasing sulfuric acid or chlorotrimethylsilane flow rate without inducing much secondary nucleation. The slow initial addition rate allows time for the crystals to grow larger, increasing the mean size. The cubic crystallization provides a less compressible filter cake, which aids in effective cake deliquoring and washing, as well as giving a more easily dried product with fewer hard lumps than the uncontrolled or constant addition rate crystallized product.

Crystals of other salts of the PPAR α/γ dual agonist free base B which may be prepared herein in accordance with the present invention include the salts of sulfuric acid, hydrobromic acid, and the like.

As indicated, the process of the invention is applicable to salt formation reactions that can use cubic addition techniques for controlled crystallization and particle size control. Examples of such salt forming reactions which can be carried out in accordance with the present invention are as follows:

Pyrrolotriazine Compound (for Treating p38 Kinase Related Diseases Such as Rheumatoid Arthritis)

Free Base I+methanesulfonic acid→mesylate salt of Free Base I 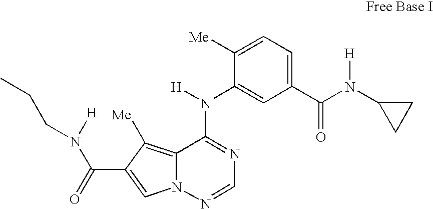
   (as disclosed in U.S. Patent No. WO 2004/043912)
   Clopidogrel (for Inhibiting Formation of Blood Clots)
Clopidogrel+H₂SO₄→Sulfate salt of clopidogrel
Clopidogrel+HCl→HCl salt of clopidogrel
Clopidogrel+HBr→HBr salt of clopidogrel 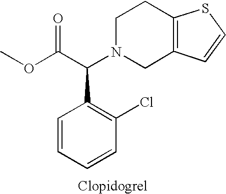
   (as disclosed in U.S. Pat. No. 4,847,265)
   Fused Pyridopyridazine Inhibitor Compound (for Treating Sexual Dysfunction)
Free Base II+methanesulfonic acid→mesylate salt of Free Base II
Free Base I+HCl→hydrochloride salt of Free Base II
Free Base II+HBr→HBr salt of Free Base II
Free Base II+H₂SO₄→sulfate salt of Free Base II 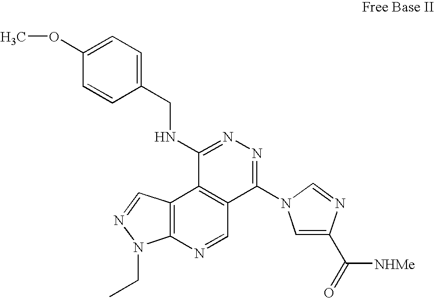
   (as disclosed in U.S. Pat. No. 6,316,438)
   PPAR α/γ Dual Agonist Compounds (for Use in Treating Type II Diabetes or Dyslipidemia)
PPAR acid+NaOH→sodium salt of PPAR acid
PPAR acid+KOH→potassium salt of PPAR acid
PPAR acid+amino acid→amino acid salt or complex of PPAR acid 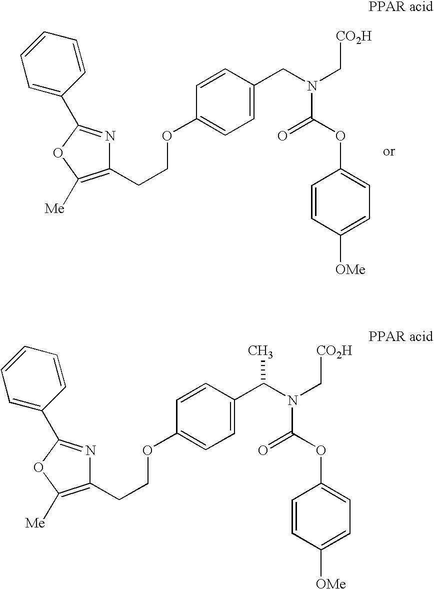
   (as disclosed in U.S. Pat. No. 6,414,002)
   Combretastatin Prodrug (for Use in Cancer Treatment)
Combretastatin+Tris(hydroxymethyl)aminomethane→TRIS salt of combretastatin
Combretastatin+L-Histidine→L-Histidine salt of combretastatin
Combretastatin+NaOH→sodium salt of combretastatin 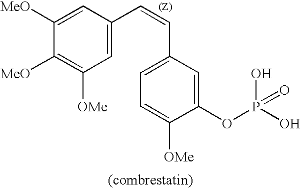
   (as disclosed in U.S. Pat. No. 6,670,344 B2)

The crystals of pharmaceuticals produced in accordance with the process of the invention may be formulated into pharmaceutical compositions for oral administration by combining the active ingredient with solid carriers, if desired granulating a resulting mixture, and processing the mixture, if desired or necessary, after the addition of appropriate excipients, into tablets, dragée cores, capsules or powders for oral use. It is also possible for the active ingredients to be incorporated into plastic carriers that allow the active ingredients to diffuse or be released in measured amounts.

The bulking agents or fillers will be present in the pharmaceutical compositions of the invention in an amount within the range from about 0.5 to about 95% by weight and preferably from about 10 to about 85% by weight of the composition. Examples of bulking agents or fillers suitable for use herein include, but are not limited to, cellulose derivatives such as microcrystalline cellulose or wood cellulose, lactose, sucrose, starch, pregelatinized starch, dextrose, mannitol, fructose, xylitol, sorbitol, corn starch, modified corn starch, inorganic salts such as calcium carbonate, calcium phosphate, dicalcium phosphate, calcium sulfate, dextrin/dextrates, maltodextrin, compressible sugars, and other known bulking agents or fillers, and/or mixtures of two or more thereof, preferably lactose.

A binder will be optionally present in the pharmaceutical compositions of the invention in an amount within the range from about 0 to about 20% weight, preferably from about 1 to about 10% by weight of the composition. Examples of binders suitable for use herein include, but are not limited to, hydroxypropyl cellulose, corn starch, pregelatinized starch, modified corn starch, polyvinyl pyrrolidone (PVP) (molecular weight ranging from about 5,000 to about 80,000, preferably about 40,000), hydroxypropylmethyl cellulose (HPMC), lactose, gum acacia, ethyl cellulose, cellulose acetate, as well as a wax binder such as carnauba wax, paraffin, spermaceti, polyethylenes or microcrystalline wax, as well as other conventional binding agent and/or mixtures by two or more thereof, preferably hydroxypropyl cellulose.

The disintegrant will be optionally present in the pharmaceutical composition of the invention in an amount within the range from about 0 to about 20% by weight, preferably from about 0.25 to about 15% by weight of the composition. Examples of disintegrants suitable for use herein include, but are not limited to, croscarmellose sodium, crospovidone, potato starch, pregelatinized starch, corn starch, sodium starch glycolate, microcrystalline cellulose, or other known disintegrant, preferably croscarmellose sodium.

The lubricant will be optionally present in the pharmaceutical composition of the invention in an amount within the range from about 0.1 to about 4% by weight, preferably from about 0.2 to about 2% by weight of the composition. Examples of tableting lubricants suitable for use herein include, but are not limited to, magnesium stearate, zinc stearate, calcium stearate, talc, carnauba wax, stearic acid, palmitic acid, sodium stearyl fumarate or hydrogenated vegetable oils and fats, or other known tableting lubricants, and/or mixtures of two or more thereof, preferably magnesium stearate.

Capsules are hard gelatin capsules and also soft, sealed capsules made of gelatin and a plasticizer, such as glycerol or sorbitol. The hard gelatin capsules may include the active ingredient in the form of granules, for example with fillers, such as lactose, binders, such as starches, crospovidone and/or glidants, such as talc or magnesium stearate, and if desired with stabilizers. In soft gelatin capsules the active ingredient is preferably dissolved or suspended in suitable oily excipients, such as fatty oils, paraffin oil or liquid polyethylene glycols, it likewise being possible for stabilizers and/or antibacterial agents to be added.

The following Examples represent preferred embodiments of the invention.

### EXAMPLES

The following Examples represent preferred embodiments of the invention.

### Example 1

### 1-[4-(Pyridin-2-yl)phenyl]-5(S)-2,5-bis{[N-(methoxycarbonyl)-L-tert-leucinyl]amino}-4-(S)-hydroxy-6-phenyl-2-azahexane, Bisulfate Salt (Form A) (Atazanavir Bisulfate—Form A)

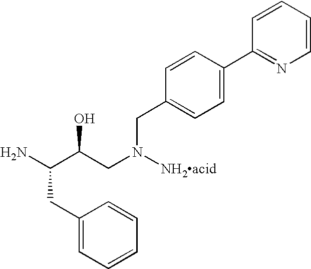

### (1-[4-(Pyridin-2-yl)phenyl]-5 (S)-2,5-bis [tert-butyloxycarbonyl)amino]-4(S)-hydroxy-6-phenyl-2-azahexane.3HCl (Triamine.3HCl Salt))

To a 1000 mL, 3-neck, round-bottom flask fitted with mechanical stirrer, nitrogen inlet and temperature probe was added the protected triamine 1-[4-(pyridin-2-yl)phenyl]-5(S)-2,5-bis[tert-butyloxycarbonyl)amino]-4(S)-hydroxy-6-phenyl-2-azahexane 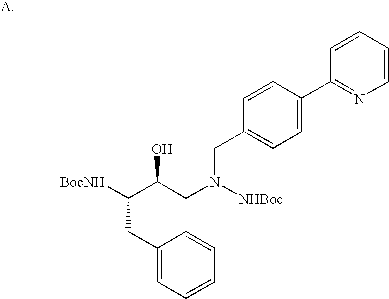
(100 g, 0.178 mol), and CH₂Cl₂ (500 mL; 5 mL/g of protected triamine input) (prepared as described in Z. Xu et al., Process Research and Development for an Efficient Synthesis of the HIV Protease Inhibitor BMS-232,632, Organic Process Research and Development, 6, 323-328 (2002)) and the resulting slurry was agitated while maintaining the temperature at from about 5 to about 22° C.

Concentrated hydrochloric acid (68 mL, 0.82 mole, 4.6 eq.) was added to the reaction mixture at a rate such that the temperature of the reaction mixture remained between 5 and 30° C. The reaction mixture was heated to 30 to 40° C. and agitated until the reaction was judged complete by HPLC assay.

Water was added (70-210 mL, 0.7-2.1 mL/g protected triamine input) to the reaction mixture, the reaction mixture was agitated for 15 minutes and the phases were allowed to separate. The upper, product (triamine.3HCl salt)-rich aqueous oil was transferred to an addition funnel.

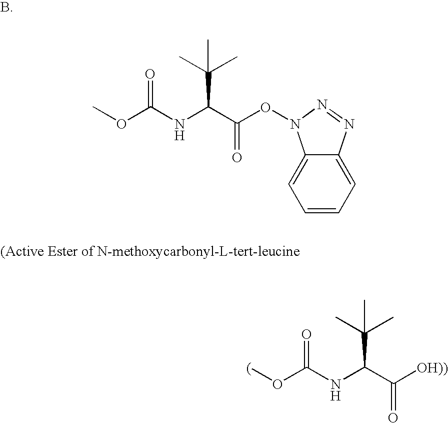

To a 3000 mL, 3-neck round bottom flask fitted with mechanical stirrer, addition funnel, nitrogen inlet, and temperature probe was added N-methoxycarbonyl-L-tert-leucine (77.2 g, 0.408 mol, 2.30 eq.), 1-hydroxybenzotriazole (HOBT) (60.8 g, 0.450 mol, 2.53 eq.), and N-ethyl N′-dimethylaminopropyl carbodimide (EDAC) (82.0 g, 0.430 mol, 2.42 eq.), followed by CH₂Cl₂ (880 mL; 8.8 mL/g of protected triamine input) and the mixture was stirred at ambient temperature (18-25° C.) until formation of the active ester is complete, as judged by HPLC.

### C. 1-[4-(Pyridin-2-yl)phenyl]-5(S)-2,5-bis{[N-(methoxycarbonyl)-L-tert-leucinyl]amino}-4(S)-hydroxy-6-phenyl-2-azahexane (atazanavir free base)

Anhydrous dibasic potassium phosphate (K₂HPO₄; 226 g., 1.30 mol, 7.30 eq. wrt protected triamine) was dissolved in 1130 mL of water (11.3 mL/g of protected amine; 5 ml/g of K₂HPO₄).

The K₂HPO₄ solution was added to the active ester solution prepared in Part B. To the stirred active ester/aqueous K₂HPO₄ mixture was slowly added the aqueous solution of Part A hydrogen chloride salt over a period of 1.5 to 2.0 h while maintaining agitation and a pot temperature between 5 and 20° C.

After the addition of the solution of the Part A hydrogen chloride salt was complete, the reaction mixture (coupling reaction) was heated to 30-40° C. and agitated until the coupling reaction was judged complete by HPLC assay.

The coupling mixture was cooled to 15 to 20° C. and the lower, product rich organic phase was separated from the upper, spent aqueous phase.

The product rich organic phase was washed with 1M NaH₂PO₄ (880 mL; pH=1.5; 8.8 mL/g of protected triamine input; 5 mole eq. wrt protected triamine), the phases were allowed to separate, and the spent aqueous phase was removed.

The washed product rich organic phase was stirred with 0.5 N NaOH (800 mL; 8 mL/g of protected triamine input) until HPLC assay of the rich organic phase showed the active esters to be below 0.3 I.I. each. The phases were allowed to separate and the spent aqueous phase was removed.

The rich organic phase was washed with 5% NaH₂PO₄ (450 mL, 4.5 mL/g of protected triamine input; pH=4.3), the phases were allowed to separate and the spent aqueous phase was removed.

The rich organic phase was washed with 10 w/v % NaCl (475 mL, 4.75 mL/g of protected triamine input) and the spent aqueous phase was removed.

The concentration of title free base in solution was 120 to 150 mg/mL with an in-process calculated yield of 95-100 mol %.

### D. Solvent Exchange from CH₂Cl₂ into Acetone/N-Methylpyrrolidone

To the rich Part C free base solution in a 3000 mL, 3-neck round-bottom flask fitted with mechanical stirrer, temperature probe, and distillation condenser, was added N-methylpyrrolidone (148 mL; 1.25 mL/g of Part C free base based on in-process quantification assay). The solution was concentrated to ca. 360 mL (2.5-3.5 mL/g of Part C free base) using a jacket temperature of 70° C. or less; 500 mL of acetone (4-5 mL/g of Part C free base) was added to the concentrated solution and the mixture was distilled to a volume of about 400 mL or less.

The acetone addition and distillation were repeated until in-process assay indicated the CH₂Cl₂ level had reached the target endpoint. At crystallization volume, the CH₂Cl₂ content in the rich organic solution was 0.77 v/v %. Acetone was added to the concentrated free base solution to reach a total solution of 16 mL/g of free base. The bath temperature was maintained at 40-50° C. to prevent crystallization of free base. The solution was polish filtered through a 10-micron or finer filter while maintaining the temperature at 40 to 50° C. The polish filter was rinsed with acetone (125 mL, 1.0 mL/g of free base) and the rinse was added to the rich free base acetone/N-methylpyrrolidone solution which was used in the next step.

### E. 1-[4-(Pyridin-2-yl)phenyl]-5(S)-2,5-bis{[N-(methoxycarbonyl)-L-tert-leucinyl]amino}-4(S)-hydroxy-6-phenyl-2-azahexane bisulfate salt

About 10% (2 g) of the total charge of concentrated sulfuric acid (19 g, 1.10 eq.) was added to the free base acetone/N-methylpyrrolidone solution of Part D, while maintaining the temperature at 40-50° C., via subsurface addition.

The reaction mixture was seeded with 5.0 wt % (wrt calculated free base in solution) of bisulfate salt. The seeded mixture was agitated at 40-50° C. for at least 30 minutes during which time the bisulfate salt began crystallizing as evidenced by the mixture increasing in opacity during this time.

The remaining sulfuric acid (17.8 g) was added over ca. 5 h in five stages according to the following protocol, defined by a cubic equation, while keeping the temperature at 40-50° C.

The rate of each addition stage was determined according to the cubic equation described hereinbefore and is shown in the table below.

| Stage | mL/kg/h | mL(H₂SO₄)/h | g(H₂SO₄)/h | Duration (min) |
|---|---|---|---|---|
| 1 | 4.62 | 0.579 | 1.065 | 60 |
| 2 | 6.93 | 0.868 | 1.597 | 60 |
| 3 | 16.55 | 2.073 | 3.814 | 60 |
| 4 | 30.26 | 3.790 | 6.974 | 60 |
| 5 | 48.47 | 6.071 | 11.171 | 23 |

After addition of H₂SO₄ was complete, the slurry was cooled to 20-25° C. for at least 1 h with agitation. The slurry was agitated at 20-25° C. for at least 1 h. The bisulfate salt was filtered and the mother liquor was recycled as needed to effect complete transfer. The filter cake was washed with acetone (5-10 mL/g of free base; 1200 mL acetone). The bisulfate salt was dried at NMT 55° C. under vacuum until the LOD <1% to produce a crystalline material.

The crystalline product was analyzed by PXRD, DSC and TGA patterns and found to be (non-solvated) Form A crystals of the title bisulfate.

The crystals produced by cubic crystallization where H₂SO₄ is added at an increasing rate according to the cubic equation described above were relatively larger and more well-defined, and had a narrower particle size range and fewer fines, than crystals obtained employing constant addition rate crystallization.

The filter cake obtained using the cubic crystallization technique was less compressible than that obtained using constant addition rate crystallization, which aided in effective cake deliquoring and washing and produced a homogeneous product.

### Example 2

### Process to Crystallize PPAR α/γ Dual Agonist Salt Intermediate A for Synthesis of PPAR α/γ Dual Agonist Compound

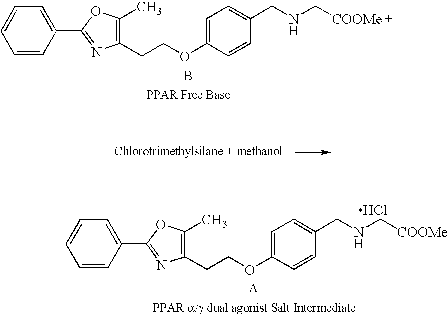

The free base solution in ethyl acetate (about 300 ml, with approximate concentration of 15 ml/g) is polish filtered. It is preferred to have a KF of ≦0.2 w/w %. Approximately 15 mL of methanol is added to the solution. The temperature is maintained between 38 and 50° C. Approximately 1-1.2 molar equiv. of chlorotrimethylsilane is added to the free base solution at an incremental rate over 3-4 hours. It is preferred to add chlorotrimethylsilane at a very slow rate initially and at increasing rate as crystallization proceeds according to the cubic equation. Seeding is preferred for better control of crystallization and can be done before chlorotrimethylsilane addition. As the free base is converted to the hydrochloride salt, crystals are formed. The addition of chlorotrimethylsilane may be done at continuously increasing rate or alternatively in several addition stages each with fixed but successively higher addition rate.

The product is collected by filtration and washed with EtOAc. The product is dried in vacuo at 50° C. PPAR α/γ dual agonist salt intermediate A is obtained as an off-white crystalline solid at 98.1-99.3% purity and 80-92 M % yield.

The salt intermediate A is used in the synthesis of an active drug substance referred to as PPAR α/γ dual agonist compound as shown in the reaction set out below and as described in U.S. provisional application No. 60/572,397 filed May 19, 2004 which is incorporated herein by reference. The PPAR α/γ dual agonist compound is useful in managing Type II diabetes and dyslipidemia. It is designed to activate peroxisome proliferator-activated receptors (PPAR) α(lipids/cholesterol lowering) and γ(insulin sensitizer).

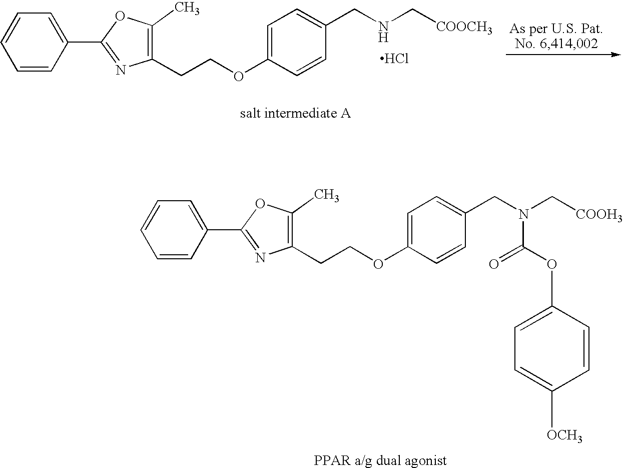

1. A process for forming crystals of a salt of a pharmaceutical by means of controlled reactive crystallization which comprises:
reacting a first reactant with increments of a second reactant added at an increasing rate according to the following equation:
$$V = {V}_{total} \times {( \frac{t}{{t}_{total}} )}^{3}$$
where
V=Volume of second reactant added up to the elapsed time period t
Vₜₒₜₐₗ=Total volume of second reactant for 100% reaction conversion
t=Elapsed time in crystallization
tₜₒₜₐₗ=Total crystallization time or total time for second reactant charging
to control the extent of reaction and crystallization kinetics and form crystals of the resulting pharmaceutical product.

2. The process as defined in claim 1 wherein the first reactant is in the form of a solution or other liquid.

3. The process as defined in claim 1 wherein the second reactant is in the form of a solution or other liquid.

4. The process as defined in claim 1 wherein the third reactant (which may or may not be needed) is optionally premixed with the first reactant or second reactant.

5. The process as defined in claim 1 wherein the first reactant is a free base of the pharmaceutical salt and the second reactant is an acid.

6. The process as defined in claim 1 wherein the first reactant is a free acid of the pharmaceutical salt and the second reactant is a base.

7. The process as defined in claim 1 further including the step of adding seeds of crystals of the pharmaceutical salt to the first reactant or to the reaction mixture of the first reactant and second reactant after a portion of the second reactant is added.

8. The process as defined in claim 5 wherein the first reactant in the form of a free base is dissolved in a solvent in which the salt of the pharmaceutical product is substantially insoluble.

9. The process as defined in claim 1 wherein the first reactant is the free base of the structure 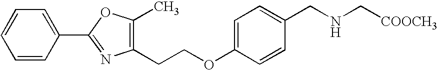
dissolved in a solvent and the second reactant is chlorotrimethylsilane and the third reactant is methanol and crystals of the HCl salt of the free base crystallize out in the solvent.

10. The process as defined in claim 9 wherein the free base is dissolved in ethyl acetate and mixed with the third reactant, methanol.

11. The process as defined in claim 9 including the step of adding seeds of the HCl salt of said free base to a solution of the free base.

12. The process as defined in claim 9 wherein from about 1 to about 1.2 molar equivalent of chlorotrimethylsilane is added to the solution of the free base incrementally.

13. The process as defined in claim 12 wherein the chlorotrimethylsilane is added at an increasing rate as crystallization proceeds.

14. A process for forming crystals of a salt of a pharmaceutical by means of controlled reactive crystallization, which comprises:
a) providing a first reactant in the form of a liquid;
b) providing a second reactant in the form of a liquid;
c) providing a third reactant (if needed) premixed with the first or second reactant;
d) adding seeds to the first reactant;
e) reacting the first reactant with a first portion of the second reactant in an amount to react with less than about 15% by weight of the first reactant; and
f) reacting the first reactant with incremental portions of the second reactant by adding the second reactant in multiple stages or at continuously varied rate to form crystals of the salt of the pharmaceutical.

15. The process as defined in claim 14 wherein the first reactant is in the form of a free base or free acid of the pharmaceutical salt and the second reactant is an acid or base.

16. The process as defined in claim 15 wherein the second reactant is added at an increasing rate according to the following equation:
$$V = {V}_{total} \times {( \frac{t}{{t}_{total}} )}^{3}$$
where
V=Volume of second reactant added up to the elapsed time period t
Vₜₒₜₐₗ=Total volume of second reactant for 100% reaction conversion
t=Elapsed time in crystallization
tₜₒₜₐₗ=Total crystallization time or total time for second reactant charging

17. The process as defined in claim 14 further including the step of adding seeds of crystals of the pharmaceutical salt to the first reactant or to the reaction mixture of the first and second reactants.

18. A process for preparing crystals of HCl salt of the structure 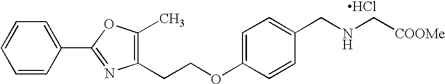
by means of controlled reactive crystallization, which comprises
a) preparing a solution of the free base of the structure 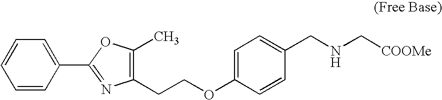
dissolved in a solvent in which the HCl salt of said free base is substantially insoluble mixed with the third reactant, methanol; and
b) reacting the free base and methanol with incremental amounts of chlorotrimethylsilane by adding chlorotrimethylsilane in multiple stages or at continuously varied rate to effect formation of crystals of HCl salt.

19. The process as defined in claim 18 wherein the free base is dissolved in ethyl acetate.

20. The process as defined in claim 18 including the step of adding seeds of the HCl salt of the free base to the solution of the free base.

21. The process as defined in claim 18 wherein the chlorotrimethylenesilane is added at an increasing rate according to the following equation
$$V = {V}_{total} \times {( \frac{t}{{t}_{total}} )}^{3}$$
where
V=Volume of chlorotrimethylsilane added per the given time period t
Vₜₒₜₐₗ=Total volume of chlorotrimethylsilane representing the 100% charge
t=Elapsed time in crystallization
tₜₒₜₐₗ=Total crystallization time or total time for chlorotrimethylsilane charging.

22. Crystals of a salt of a pharmaceutical prepared by the process as defined in claim 1.

23. Crystals of a salt of a pharmaceutical prepared by the process as defined in claim 14.

24. Crystals of the HCl salt of the structure 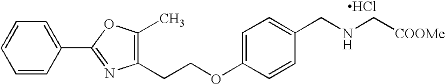
prepared by the process as defined in claim 18.
